(19) United States
(12) Patent Application Publication
(10) Pub. No.: US 2008/0200888 A1
(43) Pub. Date: Aug. 21, 2008

(54) Vaginal Discharge Collection Device

(76) Inventors: Shonta Y. Gooch, Riverdale, GA (US); Tracy H. Strozier, Riverdale, GA (US)

Correspondence Address: Shonta Y. Gooch, 584 Rountree Rd, Riverdale, GA, 30274, US

(21) Appl. No.: 11/676,386

(22) Filed: Feb. 19, 2007

(51) Int. Cl. A61F 5/44

(52) U.S. Cl. 604/330

(57) ABSTRACT

A vaginal discharge collection device is formed of an elastomeric rim and a flexible film reservoir lined with a non-toxic absorptive material. The rim may have a generally rectangular cross section and forms a collection space for collecting vaginal discharge. The rim is sufficiently resilient to provide an outward resilient force suitable to maintain the device in position within a user's vaginal canal during use. The rim and the reservoir are arranged such that compressing diametrically opposed portions to facilitate insertion of the device. The reservoir may be collapsible so as to be substantially enclosed within the rim when the device is being used. The device is constructed to be easy to use, affordable, internally comfortable, and most of all efficient.

### BACKGROUND OF THE INVENTION

There have been several failed attempts at designing an efficient internal vaginal discharge collection device. Two recent designs purportedly reducing such problems are disclosed in U.S. Pat. No. 5,295,984 (Contente et al.) & U.S. Pat. No. 5,743,893 (Kalb). The device Contente et al. comprises an elastomeric rim and a flexible film reservoir. An absorbent pad of cotton fiber or other absorbent material may be placed within the collection space defined by the rim and the reservoir. The absorbent pad may be impregnated with a drug to be delivered into the vagina. Still a number of problems, including lack of capacity, poor performance and difficulty of use, persist with this device. Furthermore the device Kalb comprises a first layer attached to a resilient rim, a second layer attached to its perimeter to the resilient rim, and also an additional layer positioned between the first layer and the second layer. A number of problems including lack of capacity, poor performance, and difficulty of use persist with this device as well.

It is thus extremely desirable to develop an improved vaginal discharge collection device that minimize or substantially eliminate such problems.

### SUMMARY OF THE INVENTION

The present invention alleviates to a great extent the disadvantages of the known vaginal discharge collection devices by providing a vaginal discharge collection device including an elastomeric rim with a generally rectangular cross section which creates a collection space for collecting vaginal discharge, and a flexible film reservoir lined with a non-toxic absorptive material, attached to the rim.

### BRIEF DESCRIPTION OF DRAWINGS

FIG. 29 is a perspective view of a preferred embodiment of a vaginal discharge collector according to the present invention.

FIG. 30 is a top view of the vaginal discharge collection device of FIG. 29.

FIG. 31 is a cross sectional view taken along the line 31-31 of FIG. 30.

FIG. 32 is a side view of the collection device of FIG. 29 in place within the vaginal canal.

FIGS. 33 and 34 are a perspective view and a top view, respectively, of the vaginal discharge collection device of FIG. 29, in a compressed configuration ready for insertion.

FIG. 35 is perspective view of the vaginal discharge collection device according to another preferred embodiment of the present invention.

FIG. 36 is a top view of the vaginal discharge collection device according to another preferred embodiment of the present invention.

### DETAILED DESCRIPTION OF PREFERRED EMBODIMENT

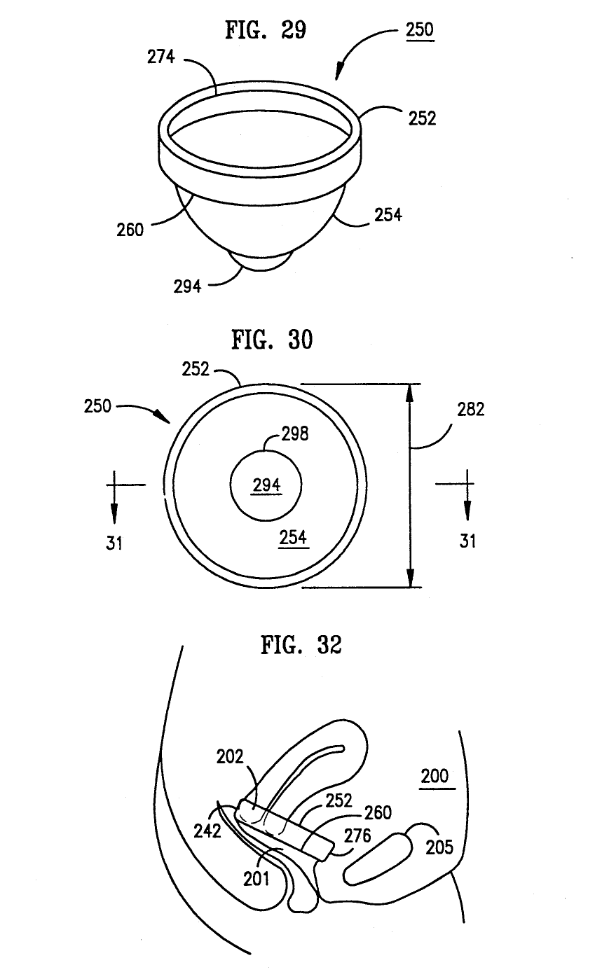

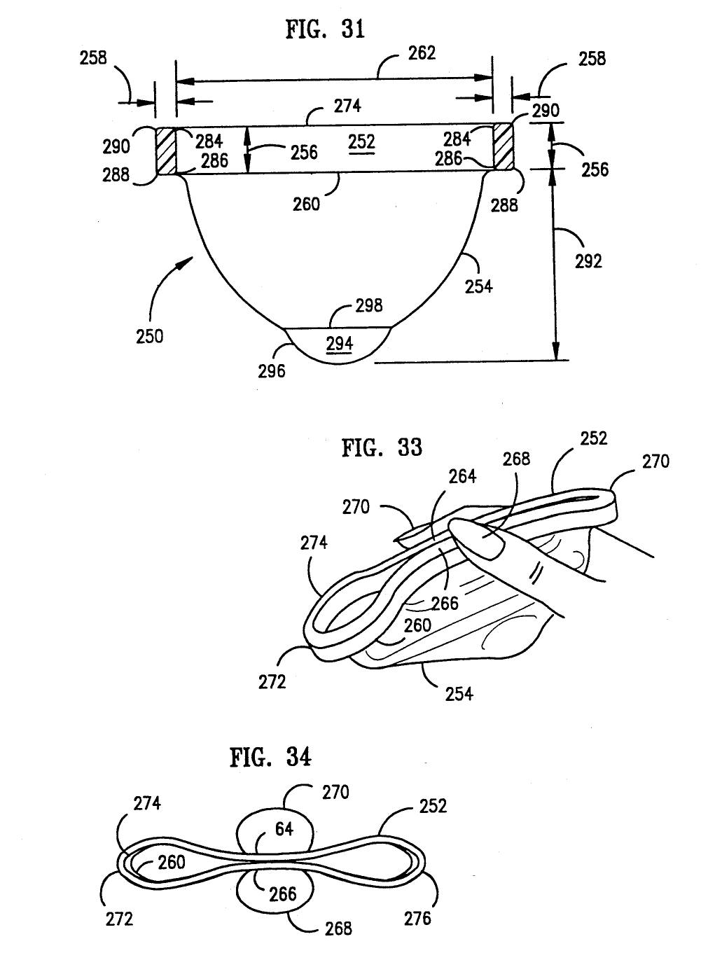

Refer now to FIGS. 29 and 30, there being shown a vaginal discharge collection device 250 constructed in accordance with another, presently preferred embodiment of the present invention. The collection device 250 is formed of a thick elastomeric rim 252 and a highly flexible reservoir 254. The reservoir 254 is formed of a thin, impervious, elastomeric film material and is sealingly connected to the rim 252. As illustrated in FIG. 31, the rim 252 has a rectangular cross section (with substantially parallel inner and outer sides, and with rounded edges), with its height 256 being substantially greater than its thickness 258.

The amount of discharge typically generated during a menstrual cycle is two to eight tablespoons (thirty to one hundred twenty milliliters). The exemplary devices 250, 10, 20, etc. illustrated in this application are designed to be worn internally for about four hours. During this four hour time period, a woman typically discharges about one teaspoon (five milliliters) of menstrual fluid, although much larger volumes of liquid may be discharged during heavy flow periods.

In operation, the device 250 is inserted into the woman's vaginal canal 201 (FIG. 32) such that portions of the rectangular rim 252 are located behind the cervix 202 and behind the pubic bone 205. In this position, the rim 252 is slightly compressed and therefore exerts an elastomeric, radially outwardly directed force on the wall of the vaginal canal 201. This force maintains the device 250 in its illustrated position during use, and prevents vaginal discharge from escaping between the rim 252 and the wall of the vaginal canal 201.

The reservoir 254 can be extended to assume a cup-shaped configuration, as illustrated in FIGS. 29 and 31. However, when the device 250 is in its collection position within the vaginal canal 201 (FIG. 32), the reservoir 254 is collapsed inwardly toward the cervix 202 by the walls of the vaginal canal 201. In this position, i.e., while discharge from the cervix 202 is being collected within the device 250, the reservoir 254 remains in its collapsed configuration essentially coplanar with the bottom edge 260 of the rim 252. Thus, in FIG. 32, the reservoir 254 is essentially hidden from view behind the rim's bottom edge 260.

Vaginal discharge is collected within a generally cylindrical space defined within the rim 252. This collection space is a virtual space in the sense that the rim 252 separates the walls of the vagina 201 to create a collection space where there is no space otherwise. In the illustrated embodiment of the invention, the inner diameter 262 of the rim 252 is approximately sixty two millimeters, and the collection volume is approximately thirty milliliters. The volume of this collection space is approximately equal to the height 256 of the rim 252 times the area surrounded by the rim 252. The reservoir 254 does not contribute significantly to the volume of the collection space, except that folds within the reservoir 254 may provide a trickling down effect, as explained below. While the device 250 is collecting vaginal discharge, the primary function of the reservoir 254 is only to seal off the bottom of the device 250. The ability of the reservoir 254 to assume a collapsed configuration allows the device 250 to be inserted and worn internally with greater comfort.

To insert the device 250 into the vaginal canal 201 adjacent the cervix 202, diametrically opposed portions 264, 266 (FIGS. 33 and 34) of the rim 252 are pressed into contact with each other between two of the user's fingers 268, 270 (which may, for example, be the user's thumb 268 and middle finger 270), such that the rim 252 assumes a figure-eight-shaped configuration. The compression applied by the fingers 268, 270 is not released (i.e., the portions 264, 266 remain in contact with each other) until a leading portion 272 of the rim 252 is in position behind the cervix 202. The compression applied by the fingers 268, 270 is then released, allowing the rim 252 to elastomerically restore itself to its initial, generally circular configuration, such that the rim 252 applies a gentle, elastomeric, radially outwardly directed force against the wall of the vaginal canal 201.

Naturally, the opposed portions 264, 266 and the leading and trailing portions 272, 276 of the rim 252 are randomly determined by the user. These portions are not defined until the user grasps the device 250 for insertion. All that is important in this regard is that the portions 264, 266 that come into contact with each other are initially approximately diametrically opposed to each other. The leading and trailing portions 272, 276 will then be defined on opposite sides of the user's fingers 268, 270.

The rectangular cross section of the rim 252 (FIG. 31) is very important. If the thick rim 252 had a circular cross section, it would tend to twist when compressed into the figure-eight-shaped insertion configuration, and further twisting could occur during insertion of the device 250 into the vaginal canal 201. Providing the rim 252 with a generally rectangular cross section therefore is very advantageous in terms of reliability and ease of insertion. Significantly, with the rectangular cross section, the device 250 can be inserted without the insertion tools that are needed with many contraceptive diaphragms.

Further, the rim 252 and the reservoir 254 can be arranged such that compressing the rim 252 into its figure-eight-shaped configuration (FIGS. 33, 34 and 35) causes the top edge 274 of the rim 252 to curve slightly downwardly, as illustrated in FIG. 35. The resulting down-dip curvature 278 of the rim's leading portion 272 makes it easier to maneuver the leading portion 272 under the cervix 202 during insertion of the device 250 into the vaginal canal 201. Preferably, the down-dip curvature 278 (i.e., the angular extent to which the leading portion 272 dips downwardly relative to a nominal plane 280 when the rim 252 is in its fully compressed, figure-eight-shaped configuration) is no less than approximately five degrees and no more than approximately fifteen degrees. If the down-dip curvature 278 is too small, some users may have difficulty moving the leading portion 272 underneath the cervix 202 during insertion. If the down-dip curvature 278 is too great, it may be difficult to move the device 250 through the vaginal canal 201.

To remove the device 250, the user inserts her finger into the vaginal canal 201 and grasps a radially inner surface of the rim 252. Preferably, the device 250 is not removed by pulling on the reservoir 254. Since the reservoir 254 is highly flexible, the user's finger can be easily pushed through the plane containing the bottom edge 260 of the rim 252, allowing the user to easily grasp the rim 252. The depth 292 of the reservoir 254 is a significant factor in the removability of the device 250. Increasing the depth 292 makes it easier for the user to insert her finger into proper position for removal of the device 250, i.e., adjacent the inner surface of the rim 252. The device 250 may also be removed by placing the finger over the top edge 274 of the rim 252 and using the finger and the thumb to grasp the rim 252 for removal.

As the device 250 is removed from the vaginal canal 201, the reservoir 254 is automatically extended to its generally cup-shaped configuration (FIG. 31) by the weight of the collected fluid. The ability of the reservoir 254 to extend in this manner minimizes the risk of spilling menstrual fluid discharge during removal and disposal of the device 250. The depth 292 of the extended reservoir 254, as measured from the bottom edge 260 of the rim 252 should be at least approximately thirty millimeters. If the depth 292 were less than approximately thirty millimeters, there may be significant spillage during removal of the device 250 from the vaginal canal 201. Also, if the depth 292 were less than thirty millimeters, some users would find it difficult to grasp the rim 252 to remove the device 250. When the depth 292 is greater than approximately thirty millimeters, the danger of spillage is substantially avoided. A depth 292 greater than approximately fifty millimeters would waste material. Excellent results are obtained when the depth 292 of the film reservoir 254 is approximately forty millimeters. Further, the device 250 may be provided in different sizes, with different depths 292. For example, the depth 292 may be thirty millimeters for a light flow product, forty millimeters for a medium flow product, and fifty millimeters for a heavy flow product.

Further, an increased depth 292 (i.e. a depth 292 greater than thirty millimeters) may provide increased volume for discharge collection through a trickling down effect. In use, the reservoir 254 is collapsed and substantially aligned with the bottom edge 260 of the rim 252. However, there may be folds within the collapsed reservoir 254 that extend downwardly beneath the edge 260, and some discharge may trickle down into such folds. Increasing the depth 292 contributes to this trickle down effect by increasing the number and length of such folds.

The device 250 has an uncomplicated construction so that it can be inexpensively mass produced and marketed. Therefore, once the device 250 has been removed from the vaginal canal 201, it can be simply thrown away and replaced by a new device 250.

The rim 252 is preferably formed of an inert thermoplastic rubber, preferably a blend of two parts of a styrenic-olefinic block copolymer marketed by Shell Chemical Company under the trademark Kraton.RTM. and one part low density polyethylene. This blended material is preferred because it is toxicologically acceptable for internal wear, readily available and economical, and readily processible. The block copolymer is particularly preferred because it has anisotropic flow properties, which means that its molecular chains can be caused to orient during plastic flow to increase stiffness perpendicular to the direction of injection molding. Without the anisotropic flow properties of the preferred material, it would be difficult to achieve the desired stiffness perpendicular to the injection molding direction. The low density polyethylene is advantageous because it increases the stiffness of the blend, improves processibility, and reduces the overall cost of the blended material.

The material of the rim 252 should be stiff enough to maintain its shape and provide the desired elastomeric self-restoring force and yet flexible enough to comfortably adjust to individual shapes. The preferred balance between stiffness and flexibility for the material of the rim 252 is obtained when the material has a Shore A hardness of approximately fifty five to approximately seventy five, preferably sixty to seventy, according to the following test method: ASTM D2240. Another important property of the preferred elastomeric material is its ability to relax and conform to the walls of the vagina as its temperature is increased from room temperature to body temperature.

The self-restoring force of the elastomeric rim 252 must be great enough to ensure that the rim 252 will expand with enough strength to form the desired seal against the wall of the vaginal canal 201, and to ensure that the device 250 will not become inadvertently dislodged. On the other hand, the self-restoring force should not be so great as to make it difficult to insert the device 250. A large self-restoring force would also make it difficult to remove the device 250. Moreover, the self-restoring force should be not be so large as to contribute to cramping or cause other discomforts. The preferred material for the rim 252 exhibits a softening effect upon exposure to the temperatures encountered in the vaginal canal 201. This advantageous property allows the rim 252 to more fully conform to the distinct shape of an individual vaginal vault once inserted. This offers greater comfort during wear as well as added protection against potential leakage during use.

Thus, the rim 252 is designed to be relatively stiff at room temperature so as to be easy to insert. The stiffness of the rim 252 decreases after insertion, as its temperature increases, making the device 250 more comfortable to wear and also easier to remove.

The rim 252 has been found to perform well in terms of self-restoring force when the rim 252 has a “compressed hoop strength” of no less than approximately two hundred and fifty grams and no more than approximately seven hundred grams, preferably no less than approximately three hundred and fifty grams and no more than approximately four hundred and fifty grams. Remarkably advantageous results are achieved when the rim's compressed hoop strength is approximately four hundred grams. As used herein, the term “compressed hoop strength” means the force needed to initially maintain the diametrically opposed portions 264, 266 of the elastomeric rim 252 in contact with each other when the rim 252 is in its figure-eight-shaped insertion configuration illustrated in FIGS. 33 and 34, with the rim 252 being at room temperature (approximately twenty three degrees Celsius).

The height 256 of the rim 252 is another important consideration. The reservoir 254 is collapsed and fulfills substantially no reservoir function while the device 250 is collecting discharge, except to seal off the bottom of the device 250. Therefore, the only way to significantly increase the device's collection volume is to increase the rim height 256. However, the rim 252 must not be too high, or it will cause discomfort. The conflicting goals of increased collection volume and increased comfort are satisfactorily balanced when the height 256 of the rim 252 is no less than approximately five millimeters and no more than about fifteen millimeters. Even better results are obtained when the rim height 256 is no less than approximately nine millimeters and no more than approximately eleven millimeters. Within this range, the rim 252 fits snugly and comfortably behind the pubic bone 205. Excellent results are achieved when the rim height 256 is approximately ten millimeters.

The thickness 258 of the rim 252 relative to the rim's height 256 is another important ergonomic consideration. Determining the most advantageous ratio between the height 256 of the rim's parallel sides to the rim's thickness 258 involves trade-offs between space utilization and the stiffness and self-restoring force of the rim 252. If the height to thickness ratio were too great, the rim 252 would either be too high (and therefore uncomfortable) or too flexible (the elastomeric self-restoring force would be too small) such that the rim 252 would tend to twist during insertion. If the ratio were too small, then the rim 252 would form an inadequately small cylindrical collection space and/or would be too thick and would also tend to twist. The best results are achieved when the rim height 256 divided by the rim thickness 258 is no less than approximately two and no greater than approximately three. The most advantageous height to thickness ratio for the preferred embodiment is two and one-half.

An advantage of the present invention is that the diameter of the device 250 does not have to be tailored to an individual user. In particular, the device 250 does not have to fit as tightly as a contraceptive diaphragm. Therefore, the device 250 can be economically manufactured in a single size and still be acceptable for most woman. A preferred outside diameter 282 for the device 250 is seventy millimeters, but satisfactory results for the single size device are achieved when the diameter 282 is no less than approximately sixty eight millimeters and no more than approximately seventy two millimeters.

It may be advantageous to manufacture the device 250 in three different sizes: (1) a junior size for teenage girls; (2) an intermediate size for nulliparous women (i.e., those who have not had a child) during the child-bearing years; and (3) a larger size for multiparous women (i.e., those who have had children). Such devices would have outer diameters 282 as follows: (1) junior—sixty to sixty five millimeters; (2) nulliparous women—sixty six to seventy four millimeters; and (3) multiparous women—seventy five to eighty millimeters. If a “one size fits all” device is desired, then the outer diameter 282 should be approximately seventy millimeters.

The rim 252 preferably has rounded edges 284, 286, 288, 290. This helps make it easy to insert the device 250 into position for use without scraping delicate tissues. Providing the rounded edges 284, 286, 288, 290 also helps avoid tissue damage during use of the device 250.

The device 250 can be formed by an injection molding process involving the following steps: injection molding the rim 252; attaching a sheet of thermoplastic elastomer to the rim 252; and vacuum thermoforming the film reservoir 254 from the sheet of elastomeric material. The above-described blended material is well suited to this injection molding process because of its anisotropic flow properties. Also, the rim 252, by virtue of its rectangular cross section, is relatively easy to injection mold. In particular, with the rectangular cross section, the rim 252 can be produced with a faster cycle time. This is because the cross section of the rim 252 is such that the injection molded material will rapidly cool and solidify. A rim with the same height 256 but with a circular cross section would take longer to solidify.

The film reservoir 254 is formed generally as thin as practically possible. Making the reservoir 254 very thin makes the device 250 easier to use and more comfortable to wear. However, if the reservoir 254 were less than about one and one-half mils thick, the reservoir 254 could cause discomfort and could be easily punctured. A preferred thickness for the reservoir 254 is about two to about six mils. If the reservoir 254 were more than about fifteen mils thick, it might not properly redeploy (extend to its FIG. 31 position) upon removal of the device 250 from the vaginal canal 201.

Advantageously, the reservoir 254 has a dimple 294. During removal of the device 250, vaginal discharge tends to flow into the dimple 294. In the illustrated embodiment, the dimple 294 will hold about one teaspoon (five milliliters) of discharge (i.e., the amount of fluid typically discharged during a four hour wear cycle). The relatively steep side walls 296 of the dimple 294 cause the discharge to remain within the dimple 294, beneath the upper edge 298 of the dimple 294. The dimple 294 forms a deep, isolated location within the device 250 during removal. The effect is to increase the extent to which discharge remains at the bottom of the device 250 during removal, reducing the likelihood of spillage. The dimple 294 also functions as a visual indicator. That is, the upper edge 298 can be easily recognized as a point of reference by the woman removing the device 250, making it easy for the woman to make a comparative determination of the amount of discharge within the device 250. The dimple 294 may also contribute to the trickling down effect by increasing the number of folds within the reservoir 254 and by increasing the volume formed by such folds.

The device illustrated in FIGS. 29-35 was constructed very well; however it has a major design flaw. The dimple 294 is not enough to prevent spillage. The non-absorptive nature of the device renders this design ineffective. The present invention is not limited to the embodiments illustrated and described in detail herein.

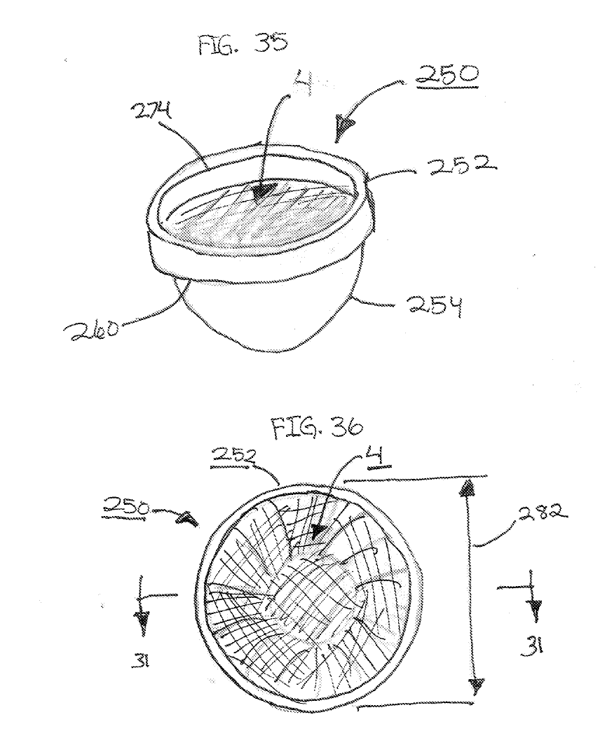

Refer now to FIGS. 36-37 there being shown a vaginal discharge collection device 1 constructed in accordance with another, presently preferred embodiment of the present invention. The collection device 250 is formed of an elastomeric rim 252 and a highly flexible reservoir 254. Advantageously, the reservoir 254 has a lining 4. The lining 4 is comprised of a non-toxic highly absorbent material (preferably of hospital grade). During use of the device 250, vaginal discharge tends to be absorbed by the lining 4. In the illustrated embodiment, the lining 4 will hold about two teaspoons (ten milliliters) of discharge (i.e., the amount of fluid typically discharged during an eight hour wear cycle). The relatively steep side walls 296 of the lining 4 cause the discharge to remain within the lining 4, beneath the upper edge 298 of the lining 4. The lining 4 forms an isolated location within the device 250 during removal. The effect is to increase the extent to which discharge remains within the device 250 during removal, reducing the likelihood of spillage. Vaginal discharge collection devices constructed in accordance with the preferred embodiment illustrated in FIGS. 29-34 have been used experimentally with excellent results.

### EXAMPLE

A study was conducted to determine the acceptability and effectiveness of the present invention. In this study, vaginal discharge collection devices like the device illustrated in FIGS. 35-36 were used over six menstrual cycles. I found the device to be the most reliable protection I have ever encountered. Moreover it was observed that satisfaction with the device increased with repeated use. After use of the device for just one menstrual cycle I discontinued use of all other sanitary protection. The devices used for this study were constructed like the device 250 illustrated in FIGS. 29-35. However each of the devices had a lining 4, and a reservoir depth 292 of approximately forty millimeters, a compressed hoop strength of approximately four hundred grams, a height 256 of approximately ten millimeters, a rim height to thickness ratio of approximately two and one-half, and an outside diameter 282 of approximately seventy millimeters. The thickness of the reservoir 254 was within the range of approximately two mils to about six mils. The lining of the reservoir 254 was a non-toxic highly absorbent hospital grade material.

The above description and drawings are only illustrative of preferred embodiments of the present invention, and it is not intended that the present invention be limited thereto. Any modification of the present invention which comes within the spirit and scope of the following claims is to be considered part of the present invention.

What is claimed is:

1. A method of using a menstrual discharge collector during a period of menstruation, said method comprising the steps of:
(a) Providing a first menstrual discharge collector including body means for providing a collection and absorption space for the collection of menstrual discharge and having an opening for the passage of said menstrual discharge into said space, and rim means for providing resilient outward holding force sufficient for holding said collector in position within a woman's vaginal canal during use, said body means having a top, said rim means having a leading portion and a trailing portion, said rim means being affixed to said body means proximate said top of said body means;
(b) Positioning said discharge collector in said position such that said leading portion of said rim means is located at a rearward location behind the woman's cervix, such that said trailing portion of said rim is located between said leading and trailing portions of said rim means;
(c) Holding said collector in said position, with a substantially looser fit than would be needed to inhibit the passage of sperm around said rim means, by applying said resilient outward holding force against the walls of the woman's vaginal canal;
(d) While holding said collector in said position, collecting a first volume of said menstrual discharge in said collection space by allowing said discharge to flow through said opening and into said space;
(e) Subsequently, disposing of said vaginal discharge collector together with said first volume of said menstrual discharge;
(f) Providing a second menstrual discharge collector including body means for providing a collection space for the collection of said collection space of said collector, and rim means for providing a second resilient outward holding force sufficient for holding said second collector in said position within the woman's vaginal canal during use, said body means of said second collector having a top, said rim means of said second collector having a leading portion and a trailing portion, said rim means of said second collector being affixed to said body means of said second collector proximate said top of said body means of said second collector;
(g) Positioning said second discharge collector in said position such that said leading portion of said rim means of said second collector is located at the rearward location behind the woman's cervix, such that said trailing portion of said rim means of said second collector is located at the forward location behind the woman's pubic bone, and such that the woman's cervix is located between said leading and trailing portions of said rim means of said second collector;
(h) Holding said second collector in said position, with a substantially looser fit than would be necessary to inhibit the passage of sperm around said rim means of said second collector, by applying said second resilient outward holding force against the walls of the woman's vaginal canal;
(i) While holding said second collector in said position, collecting and absorbing a second volume of said menstrual discharge in said collection space of said second collector by allowing said discharge to flow through said opening of said second collector and into said collection space of said collector; and
(j) subsequently, disposing of said second discharge collector together with said second volume of said menstrual discharge; and
wherein said steps of positioning and disposing of said first discharge collector and said steps of positioning and disposing of said second discharge collector all occur during said period of menstruation.

2. The device according to claim 1, wherein said body means of said first discharge collector includes an absorptive reservoir space extending from said collection space of said first discharge collector.

3. The device according to claim 2, wherein said body means of said first collector includes a generally cup shaped main wall portion and a bubble-like protrusion, being integrally connected to said generally cup-shaped main wall portion forms said collection space of said first collector, and wherein said bubble-like protrusion forms said reservoir space of said first collector, and wherein said bubble-like protrusion is substantially smaller than said generally cup-shaped main wall portion, and wherein said second collector is substantially identical to said first collector. Wherein said bubble-like protrusion is lined with a non-toxic absorptive material.

4. The device according to claim 1, wherein said first collector includes closure means for at least partially covering said opening of said first collector for inhibiting said discharge from exiting said collection space of said first collector.

5. The device according to claim 1, wherein said body means of said first collector includes a flexible film reservoir lined with a non-toxic absorbent material, said reservoir being attached to said rim means.
